(19) United States
(12) Patent Application Publication
(10) Pub. No.: US 2011/0136178 A1
(43) Pub. Date: Jun. 9, 2011

(54) BASE-MODIFIED PRIMER OLIGOMERS FOR MULTIPLEX RT-PCR

(76) Inventor: Olfert Landt, Berlin (DE)

(21) Appl. No.: 12/863,490

(22) PCT Filed: Jan. 9, 2009
(86) PCT No.: PCT/DE09/00083

§ 371 Date: Sep. 2, 2010

(30) Foreign Application Priority Data
Jan. 19, 2008 (DE) 10 2008 005 667.7

(51) Int. Cl. C12P 19/34; C12N 9/10

(52) U.S. Cl. 435/91.1; 435/193

(57) ABSTRACT

The invention relates to a method for the amplification of nucleic acid sequences by enzymatic DNA polymerase activity, wherein synthetic oligodeoxynucleotide compounds are used as primers, comprising sequences having the formula (I), wherein the sequence SeqA-Ni-SeqC has at least a length of 8 nucleotides and can hybridize with one of the nucleic acid sequences to be amplified, SeqA and SeqC are sequences of naturally occurring deoxyribonucleotide building blocks or the synthetic analogs thereof, N1 is a phosphoribonucleotide group covalently bound to R1 or a nucleotide analog covalently bound to R1, R1 is a molecule group, comprising at least ten atoms having an atomic weight of twelve and higher, and to reaction mixtures for carrying out the method.

The present invention relates to the field of enzyme catalyzed amplification of nucleic acids, especially means and methods for use in multiplex RT PCR.

Polymerase chain reaction (PCR) is a method to amplify, detect and determine the amount of nucleic acids in a sample.

Conventional PCR methods detect sequences made of deoxyribonucleic acid (DNA); ribonucleic acid sequences must be reverse transcribed (RT) into cDNA before being amplified in a PCR reaction. The combination of reverse transcription and PCR is called RT-PCR. The transcription into DNA is carried out by a RNA-dependent DNA polymerase (reverse transcriptase). The most common reverse transcriptases (below also: RT-enzymes) used are those from moloney murine leukemia virus (MMLV) and avian myeloblastosis virus (AMV).

Nowadays, both PCR and RT-PCR are widely used in the detection and quantification of pathogenic organisms, of which RNA-viruses represent an important group. Additionally RT-PCR is used to detect and amplify mRNa of non-viral origin. The transcription activity of a cell can correlate with its status regarding, for example, its differentiation, or with a previous drug treatment or with a disease pattern. One example would be the amplification of chimeric mRNA-transcripts in order to detect chromosomal translocation in certain cases of leukemia.

The transcripts are amplified in a quantitative RT-PCR, usually they are analyzed in comparison to the amount of transcript from a status-independent reference gene (“housekeeping”-gene).

For the detection of pathogens, a second PCR is carried out preferentially as a control reaction. If the test result for pathogen detection is negative, the control reaction is meant to serve as proof that the reaction parameters were approptriate to generate a PCR product in the probe (internal PCR control, IPC).

Also for analysis of the transcription activity of cells, a control is always performed, either in order to monitor the PCR conditions or in case of a quantification in order to compare the results to a quantity standard.

The most elegant solution is to run the controls described in the previous paragraphs within the same reaction set-up as the samples; this is referred to as a duplex-PCR. If more controls or more primers for further target sequences are present, such reactions are referred to generally as multiplex-PCR.

The RT-PCR can either be carried out in two steps (2-step RT-PCR) or in a combined step (one-step-reaction, 1-step RT-PCR). Reverse transcriptase and DNA polymerase have their optimal working conditions at different concentrations of salts and additives (buffer conditions) as well as at different temperatures. Therefore, the 1-step RT-PCR will always be a compromise, which goes along with a decreased effectiveness of either one or both of the reactions. This can affect either the specificity or the processivity of the two reaction steps. Nevertheless, the 1-step reaction is widely preferred, as it requires significantly less manual working steps, it is easy to automate and faster than the 2-step reaction, and the risk for contamination of the sample is diminished.

The 1-step reaction often shows a decreased sensitivity or requires more thermocycles, which is due to the low processivity of the DNA polymerase. The difference in sensitivity of the method can be several orders of magnitude when detecting small amounts of RNA. This can be shown experimentally by comparing the detection limit of a DNA-PCR of a specific DNA sequence with or without the RT-step.

In RT-PCR, primer mixes are used frequently, for example in order to detect several genes or virus variants concomitantly, but in any case for the amplification of the comprised control or reference PCR. PCR reactions in which more than one primer pair is present are called multiplex reactions, multiplex PCR or, where applicable, multiplex RT-PCR. In the present application, the term “multiplex” is used when two, three, four or more primer pairs are present.

It has been shown in many cases that even if appropriate matrix sequences are present in the reaction solution, multiplex reactions did not give the expected result (false negative result) when the number of primers used in the reaction is increased. In some cases even the presence of only a third primer prevented DNA amplification. Many of the published multiplex methods are the result of extensive primer searches by trial and error.

To the inventor's knowledge, a conclusive analysis of the reasons for such failure of many multiplex primer mixes in RT-PCR has not been published. Older analyses exist regarding the phenomenon of “primer dimer” formation in conventional PCR, as well as approaches for solving the problem.

Departing from the presented state of the art, the objective of the invention defined herein is to provide means and methods for carrying out multiplex RT-PCT reactions with complex primer mixes, allowing for highly sensitive and selective amplification of the sequences detected by the employed primer pairs.

This objective is attained by the subject-matter of the independent claims.

According to one aspect of the invention a method for enzymatic amplification of nucleic acids is provided, in which more than two synthetic oligodeoxynucleic compounds are used as primers. At least one of these primers comprises a sequence that can be described through the formula

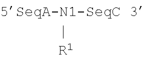

The sequence SeqA-N1-SeqC has a minimum length of 8 nucleotides, rather 15 to 25 nucleotides, and is able to hybridize with one of the nucleic acid sequences to be amplified. SeqA and SeqC are sequences of naturally occurring deoxyribonucleic elements or their synthetic analogs. SeqA 0 has at least 40 nucleotides; SeqC is a sequence of 1 to 20 nucleotides in length. SeqC has a preferred length of 1 to 10 nucleotides, a length of 1 to 5 is even more preferred. According to the most preferred embodiment, SeqC has a length of one nucleotide.

N1 is a phosphoribonucleic moiety covalently bound to R1, or a nucleotide analog covalently bound to R1, R1 is a molecular moiety comprising ten atoms of a atomic weight of 12 or more.

According to a preferred embodiment of the invention, at least three different primers are used comprising at least one sequence, which can be described by the formula

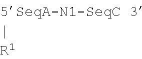

It is understood that the sequence sections SeqA and SeqC can be different for each of the different primers, similarly the modified nucleotides or analogs, which are represented by N1, or their modifications.

According to another preferred embodiment of the invention, at least four different primers are used for the amplification of at least two different nucleic acid sequences. Preferably two or three different primer pairs are used, in order to detect at least three or more different nucleic acid sequences to be amplified. It is understood that not all primer pairs must consist of different primers, thus two different “forward” primers but the same “reverse” primer can be used for the amplification or detection of two different sequences.

According to another embodiment of the invention SeqA comprises at least one phosphoribonucleic moiety covalently bound to a molecular moiety R2 or a nucleotide analog covalently bound to R2. R2 shows the same characteristics as R1, whereas R1 and R2 can be different within one molecule. A primer used in a method or reaction mix according to the invention can therefore contain several covalently modified bases.

R1 or R2 or both comprise preferably one to five rings with four to seven atoms as ring members per ring. Two to four rings are more preferred. Similarly preferred are embodiments, in which R1 or R2 or both comprise at least fifteen atoms with an atomic weight of 12 or more. R1, R2, or both can for example be methylene blue or methyl orange.

Preferably, an enzymatic reverse transcription activity is used for polymerization of deoxyribonucleic acid from a ribonucleic acid template in a reaction step preceding the amplification. This reverse transcription activity can for example comprise the activity of the reverse transcriptase from moloney murine leukemia virus (MMLV) and/or avian myeloblastosis virus (AMV).

According to another aspect of the invention, a reaction mix for enzymatic amplification of nucleic acid sequences is provided, comprising more than two synthetic oligodeoxynucleotide compounds as primers, which are denoted by the above mentioned elements of the primers used in the method according to the invention. A primer mix is provided as well, which can be used in a method according to the invention, or to produce a reaction mix according to the invention, and which contains at least three primers, of which at least one is modified according to the above mentioned characteristics. Primer mixes containing at least two modified primers are preferred.

According to the investigations made in the context of the present application, the lack of efficiency in RT-PCR with complex primer mixes is a result of a DNA dependent polymerase activity of the reverse transcriptase, a side activity of the enzyme. The primers anneal to each other during the relatively low temperatures at the RT step and are then elongated at the 3′ end, even if they do not match exactly. The resulting primer dimers do not hybridize with sufficient selectivity to the target DNA, and therefore do not serve as primers for the target sequence anymore.

The basis of the solution of the problem underlying the present invention is the inhibition of the DNA dependent DNA polymerase activity of the RNA dependent reverse transcriptase, which is innate to the RT enzyme. The inventive approach presented here differs significantly from the methods in the state of the art regarding both the molecule targeted for inhibition, and the choice of methodology. While the publications of the state of the art aim at reducing base pairing in incorrect hybrid bonds between non-canonical base pairs, the present invention inhibits the RT dependent primer dimer formation by inhibiting the RT itself.

Non-canonical base pairs are those, which do not correspond to the conventional pairing A-T (A-U) and G-C.

While searching for appropriate reaction parameters for complex multiplex reactions, it was surprisingly found that certain chemical modifications of the bases reduce the primers' affinity towards the RT enzyme while showing no significant decrease in their activity as a primer in the PCR amplification.

Key idea of the present invention is the use of primer mixes in multiplex RT PCR reactions. A primer mix should consist of several, different, DNA-oligomers which are essentially homogeneous with regard to their sequence and their constitution (one “primer”). Whenever one or more “primers” are mentioned below, it does not mean—as long as the context provides no compellingly different meaning to the skilled person—single molecules but rather one or more essentially homogeneous populations of oligomeric DNA molecules that serve as primers. “Modified primers” comprise modifications compared to DNA molecules known in nature. Modified primers in the sense of the invention comprise modified bases close to their 3′ end, which are covalently bound to large organic moieties.

The statements below refer to a method, where the reaction progress is monitored directly by hydrolysis of fluorescently labeled target-specific oligonucleotides. This method is known to the skilled person as real-time PCR or TaqMan-PCR (see EP0543942B1 and family members). Parameters of the TaqMan reaction are the level of fluorescence, which will increase during the reaction progress, as well as the threshold ct, which signalizes a positive result (target sequence is present).

The present invention is not restricted to use in the TaqMan PCR assay, but can be used with any kind of PCR, as long as it comprises the use of several primers in one reaction mix (multiplex PCR).

The inventive effect in the context of the present description, is a reaction progress that is altered when using of inventive reaction mixes of modified oligodeoxynucleotide primers in comparison to the use of unmodified primers.

This inventive effect can consist in an increase of the level of relative fluorescence in the saturation phase of a reaction, relative to an unmodified primer used as a comparison for a given number of template molecules and at otherwise unchanged reaction conditions. Furthermore, the inventive effect can be regarded as a decrease or lowering of the number of reaction cycles before reaching the threshold (ct), at which the reaction can be assessed as positive. Similarly, the inventive effect can be an improvement of the reaction according to the criteria mentioned above in the presence of various competing or non-competing primers, or a combination of all the criteria mentioned above.

Further investigation revealed that the inventive effect occurs when the primer modification of at least one base close to the 3′ end with a large organic moiety has a certain distance to the base and a certain size.

Modified primers can be produced, for example, by means of solid state synthesis through assembly of derivatized phosphoamidite bases. Commercially available are modifications at the C-5 and C-6 carbon atom of the pyrimidin base phosphoamidite elements.

A simple aliphatic moiety with amino function at the C-6 carbon atom of the pyrimidin base, which can be bought as a phosphoamidite element (“aminolinker”, see for example. Glen Research Corporation, Virginia, USA, product 10-1039-90) (formula (1)) or other aliphatic groups in the 5-position of a deoxythymidine base do not show an effect.

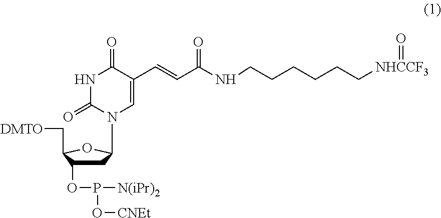

Primers with bases at or close to the 3′end, which are modified with small heterocycles such as for example methyl orange (see formula (2), available from Glen Research Corporation, Virginia, USA, product 10-1058-95), do show the inventive effect.

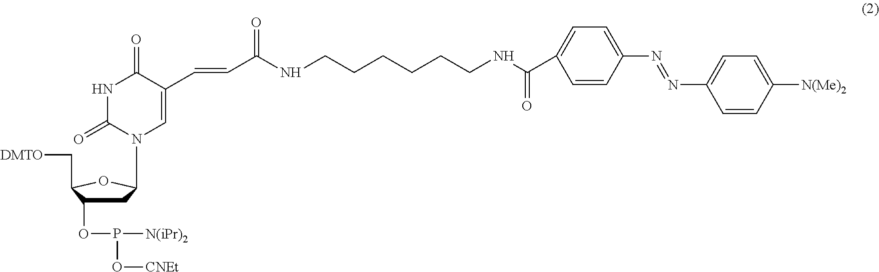

Exemplary are oligodeoxyribonucleoside primers in which nucleosides on the basis of compounds such as shown in formulas (1) or (3) were assembled as chain links close to or at the 3′ end of the primer during synthesis, wherein the compounds shown in (3) to (5) are modified with organic moieties comprised of at least 10 carbon and/or hetero atoms. Preferred modifications of primer bases are those at the pyrimidine ring's 5-position or the purine ring's 8-position.

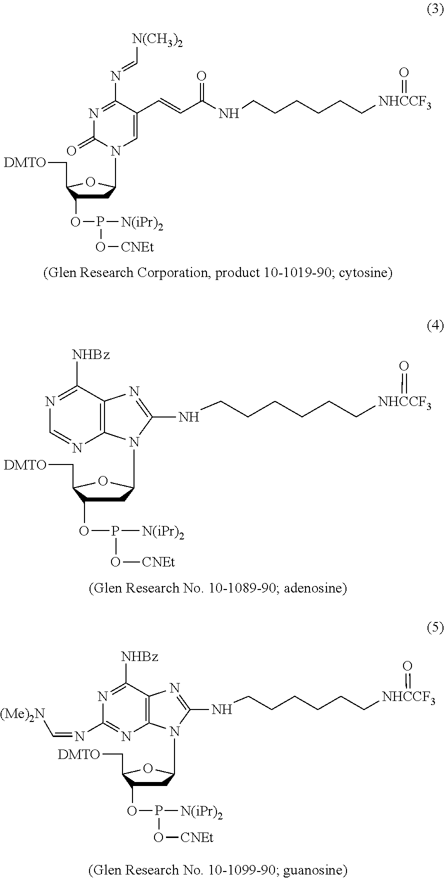

Preferred are modifications by organic moieties, which have an aliphatic linker molecule that comprises two to ten carbon or hetero atoms in the chain, as well as modifications by organic moieties which comprise at least ten carbon or hetero atoms. Preferably, these at least ten carbon and/or hetero atoms form one or more five or six ring systems. Hetero atoms can be, inter alia, Nitrogen, Oxygen or Sulfur.

Especially preferred are modifications with linkers between 3 and 6 atoms and ring systems of three or more rings.

Further base modifications can be used that are known to the person skilled in the art and can be obtained without an inventive step, such as different bridging molecules than those used in the examples, and modifications at positions different from those mentioned.

The strength of the inventive effect depends on the type and size of the modification. The above mentioned amino linkers without further attached groups do not show much effect. If the mentioned bases are modified with hetero cycles such as methyl orange (Glen Research product-No. 10-1058-95), an increase in signal intensity or a reduction of cycles in the RT-PCR occurs. Systems consistent of at least three rings which are attached to the base through a linker show the inventive effect. The dyes carboxy fluorescein, tetramethylrodamine (TAMRA), rhodamine LightCycler Red640 as well as the smaller methylene blue all show the inventive effect.

Preferred are small aliphatic bridges or linker of one to twenty, especially from two or four to thirteen, further preferred from two or three carbon atoms to eight carbon and hetero atoms within the direct chain between the ring system inventively attached to the base; especially preferred is the acrylimido-aminoethyl bridge between base and modification, which establishes the bridging via seven atoms. If a thirteen atom linker is used for the same dyes, a smaller effect can be observed, which might still be sufficient for the respective task.

Modifications by molecules containing two to six ring systems are preferred, especially preferred are modifications by one to five, preferably two, three or four ring systems condensed or connected by bridges, said ring systems containing 5-6 carbon or hetero atoms, especially preferred are aromatic systems. Considerably larger groups block the primers during PCR amplification, presumably because of interactions with the DNA polymerase.

Especially preferred are modifications by dyes such as methylene blue, fluorescein, TAMRA, methyl orange, which all share the characteristics of consisting of two to five six-rings, either condensed or attached to each other via rotatable axes.

In principle, every base within a primer can be modified. A relative proximity to the 3′ end of the primer is of advantage. Rhe bases in position 1 to 6 relative to the 3′ end are preferred (the base which is the 3′ end would have the number 0 in this nomenclature). According to the invention, the inventive effect can still be seen when the base at position 14 is modified. The inventive effect decreases with increased distance to the 3′ end, therefore a relative proximity of the modification to the 3′ end is indicated, yet there might be reasons within the structure of the sequence to move further away from the 3′ end.

Most preferred is the use of a modification at position 1 of the sequence.

A primer molecule for the use in a method or in a primer mix according to the invention, has a length of 10 to 50 nucleotides. Preferred are primers with a length of 10 to 40, especially with 15 to 30 nucleotides.

In the examples, naturally occurring base elements for the production of primers according to the invention are preferably used. Synthetic base analogs such as locked nucleic acid, peptide nucleic acid and other analogues, which use not naturally occurring backbone group as analogues of the natural sugar in DNA, also can be considered for practicing the invention.

According to one embodiment of the present invention, primer mixes for the diagnosis of viral infections are supplied. The invention has shown to be of use for the production of primers and analysis kits used to diagnose influenza, especially in the context of the currently growing relevance of the H5N1 subtype, as well as HIV and HCV. Furthermore this invention can be used for diagnosis of the earlier mentioned viral infections.

According to one embodiment of the invention RNA viruses, especially retro viruses such as HIV; coronavirus, which includes the pathogen for SARS and other veterinarily important diseases; rhabdovirus, which includes the pathogen for rabies; picornavirus, which includes the pathogen for polio; filovirus, which includes the pathogens for Ebola and Marburg fever; flavivirus, which includes the pathogen for hepatitis C, yellow fever and dengue; reovirus, which includes the pathogens for the classical cold; rotavirus; influenza or the pathogen for mumps are detected separately or in combination by multiplex RT-PCR.

Furthermore, according to yet another embodiment of the invention, detection of pathogen specific mRNA of viruses in general, also from DNA viruses, as well as from bacteria and parasites via multiplex RT-RNA can be achieved, especially in order to determine the activity or infectious potential of the pathogen.

According to an embodiment of the invention, reaction mixes are provided, which comprise several primer pairs for the detection of viral and other target RNAs in one sample. Reaction mixes for the detection of viral RNA according to the invention are mixes that consist of several primer pairs appropriate for multiplex RT-PCR.

According to another embodiment of the invention these reaction mixes contain different synthetic oligodeoxynucleotide compounds as primers, where at least one preferably several or all of said synthetic oligodeoxynucleotide compounds can be described by the following formula

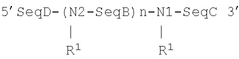

wherein
SeqD, SeqB and SeqC are sequences of naturally occurring deoxyribonucleic elements or their synthetic analogs, whereby a mixture of deoxyribonucleic acid elements and their synthetic analogues should also be covered,
n means 0 to 3, preferably 0 or 1,
SeqD can have a length of 0 to 40 nucleotides,
repeats for SeqB for n>1 can have different sequences and lengths of 0 to 25 nucleotides within the oligodeoxynucleotide compound,
SeqC has a length of 1 to 20 nucleotides, preferably 1 to 12,
N1 and N2 are base elements or synthetic base analogs modified either at an atom constituting the six-ring of the pyrimidine or the five-ring of the purine,
R1 and R2 are a molecular non-fluorescent moieties covalently, preferably via an aliphatic bridge, bound to N1 or N2, which comprise at least ten atoms of an atomic weight of twelve or more, preferably comprising at least two to five aromatic rings.

For n=0 SeqD equals SeqA according to the above mentioned definition, whereby SeqA does not contain a modification. For n>0 SeqA consist according to the above mentioned definition of the sequences SeqD-(N2-SeqB)n.

Reaction mixes and methods are preferred, in which at least three different synthetic oligodeoxynucleotide compounds as primers are used; even more preferred are those which contain at least four different synthetic oligodeoxynucleotide compounds as primers for the amplification of at least two different nucleic acid sequences.

According to a preferred embodiment SeqC has a length of 1 to 10 nucleotides and n equals 0.

The invention will be characterized in more detail by the following examples.

### EXAMPLE 1

### Influenza H5N1 Amplification H5 Gene

### Primer Synthesis

Primers were synthesized with an amino-modified d-thymidine base linked to a C2 linker, C6 linker, a dT-Fluorescein or a dT-C6-Dabcyl (all previously mentioned modifications obtained from Glen Research) or a dT-BBQ (Berry & Associates Inc., Dexter, Mich., USA) at the position marked by XT, without cleavage of the terminal trityl-group on a Expedite 8900 synthesizer with standard cycle and standard phosphoramidites obtained from Proligo LLC, Boulder, Colo., USA. The protecting groups were removed at 55° C. overnight in concentrated ammonia, which was then removed using NAP-10 Sephadex gel filtration columns (GE Healthcare). The primers were purified using a Oligo-R3 HPLC column (Perseptive Biosystems Inc., Framingham Mass., USA) after the removal of the trityl group (60% acetic acid or 3% triflour acetic acid) using a SourceQ column (Amersham Pharmacia Biosciences) in 10 mM NaOH. For the synthesis of the ester-modified primers, the purified amino-modified primers were linked to NHS-Ester (TAMRA-NHS, emp Biotech GmbH, Berlin; LightCycler Red 640, Roche Diagnostics; methylene blue-NHS, emp Biotech) in 50% DMSO. The products were then purified using HPLC and gel filtration.

### System 1

| | | | | |
|---|---|---|---|---|
| InfA_TH5S | ATATgTgAAATCAAACAgATTAgTCC | S | 67-92 | 52.4° C. |
| InfA_TH5xA+1 | TCTgCAgCgTACCCAC T CC | A | 258-238 | 60.2° C. |
| InfA_TH5xA | TTgTCTgCAgCgTACCCAC XT C | A | 258-238 | 60.2° C. |

### System 2

| | | | | |
|---|---|---|---|---|
| InfA_TH5S | ATATgTgAAATCAAACAgATTAgTCC | S | 67-92 | 52.4° C. |
| InfA_TH5xR+1 | TggATTCTTTgTCTgCAgCg T A | A | 267-245 | 59.6° C. |
| InfA_TH5xR | gTggATTCTTTgTCTgCAgCg XT A | A | 267-245 | 59.6° C. |

Modification of the reverse Primer A, R at the last but one (n−1) position with:

dT-C2-Amin; dT-C6-Amin; dT-C2-Fluorescein; dT-C2-TAMRA; dT-C2-LC Red 640; dT-C2-methylene blue; dT-C6-methyl orange (Dabcyl); dT-C6-Fluorescein; dT-C6-TAMRA (6-Carboxytetramethylrhodamin); dT-C6-LC Red 640; dT-C6-methylene blue; dT-C2-BBQ.

The reaction set up was a 1-step RT mix, consistent of FastStart DNA Master (Roche Diagnostics) for DNA amplification, which contained 0.2 u Transcriptor reverse transcriptase (Roche Diagnostics). 10 pmol primer (0.5 μM) and 3 μmol hybridization probes (0.15 μM) were used in a total volume of 20 μl. The reaction mix was incubated for 10 min, denatured for 10 min at 95° C. and then run for 55 cycles: 2 sec 95° C., 10 sec 55° C. and 10 sec 72° C. in a LightCycler 2.0 (Roche Diagnostics). A dilution series of a plasmid containing the target sequences was used as a target (GenExpress GmbH, Berlin).

### EXAMPLE 2

### Position Variation of the Modified Base by Primer Shift

Forward primer TH5S and several reverse primer, with the same base modification (dT-Fluorescein), so that the modified base is 2 to 5 bases away from 3′ end. The ct value (threshold) and the level of relative fluorescence in the saturation stage of two target concentrations, which are equivalent to 1000 and 100 genome equivalents, were determined. The sequence with a modification at the second position (TH5xA) was used as reference. The relative fluorescence was two to six times higher compared to the unmodified primer for the same target with 1000 copies; the reaction did not give a signal for the 100 copies target for two unmodified primers

| | | | ct | rel Fluor.* | |
|---|---|---|---|---|---|
| | | pos | 1E3 | 1E3; 1E2 | |
| InfA_TH5S | ATATgTgAAATCAAACAgATTAgTCC | | | | |
| InfA_TH5xA+3 | gCAgCgTACCCAC XT CCCC | -5 | 0 | 59 | 29 |
| InfA_TH5xA+3 | gCAgCgTACCCAC T CCCC | | 0 | 9 | 0 |
| InfA_TH5xA+2 | TgCAgCgTACCCAC XT CCC | -4 | 0 | 64 | 45 |
| InfA_TH5xA+2 | TgCAgCgTACCCAC T CCC | | 0 | 27 | 0 |
| InfA_TH5xA+1 | TCTgCAgCgTACCCAC XT CC | -3 | 0 | 87 | 80 |
| InfA_TH5xA+1 | TCTgCAgCgTACCCAC T CC | | 0 | 45 | 17 |
| InfA_TH5xA | TTgTCTgCAgCgTACCCAC XT C | -2 | 0 | Ref 100% | |
| InfA_TH5_A | TTgTCTgCAgCgTACCCAC T C | | 0 | 98 | 55 |

Forward primer TH5S and several reverse primer, with the same base modification (dT-Fluorescein), so that the modified base is 2 to 7 bases away from 3′ end. The ct value (threshold) and the level of relative fluorescence in the saturation stage of two target concentrations, which are equivalent to 100 and 1000 genome equivalents, were determined. Reference was the sequence with a modification at the second position (TH5xR). The relative fluorescence was two to three times higher compared to the respective unmodified primers for the same target. The primers with modifications at position −2 and −6 had an earlier ct value.

| | | | ct | rel Fluor.* | |
|---|---|---|---|---|---|
| | | pos | 1E3 | 1E3; 1E2 | |
| InfA_TH5S | ATATgTgAAATCAAACAgATTAgTCC | | | | |
| InfA_TH5xR+5 | TTTgTCTgCAgCg XT ACCCAC | -7 | 0 | 73 | 67 |
| InfA_TH5xR+5 | TTTgTCTgCAgCg T ACCCAC | | 0 | 27 | 24 |
| InfA_TH5xR+4 | CTTTgTCTgCAgCg XT ACCCA | -6 | -1 | 66 | 74 |
| InfA_TH5xR+4 | CTTTgTCTgCAgCg T ACCCA | | -1 | 39 | 35 |
| InfA_TH5xR+3 | TTCTTTgTCTgCAgCg XT ACCC | -5 | 0 | 71 | 65 |
| InfA_TH5xR+3 | TTCTTTgTCTgCAgCg T ACCC | | 0 | 56 | 33 |
| InfA_TH5_R | gTggATTCTTTgTCTgCAgCg T A | | 0 | 68 | 33 |

| | | | | |
|---|---|---|---|---|
| InfA_TH5xR | gTggATTCTTTgTCTgCAgCg XT A | -2 | -2 | Ref. 100% |

### EXAMPLE 3

### Variation of the Position in the Sequence (R)

Forward primer TH5S and reverse primer TH5_R with internal modified dT bases at different positions from −2 to −14. All modified primers show a signal approximately 50% higher than the modified references for the target with 1000 copies and up to 450% higher for the target with 100 copies. The double modified primer THG5xR** showed a 550% higher signal. The ct values were 2 cycles lower for all modified primers.

| | | | ct | rel Fluor.* | |
|---|---|---|---|---|---|
| | | pos | 1E3 | 1E3; 1E2 | |
| InfA_TH5S | ATATgTgAAATCAAACAgATTAgTCC | | | | |
| InfA_TH5_R | gTggATTCTTTgTCTgCAgCg T A | | 0 | 68 | 33 |
| InfA_TH5xR | gTggATTCTTTgTCTgCAgCg XT A | -2 | -2 | Ref. 100% | |
| InfA_TH5xR9 | gTggATTCTTTgTCTgCAgCg T A | -9 | -2 | 103 | 148 |
| InfA_TH5xR13 | gTggATTCTTTgTCTgCAgCg T A | -13 | -2 | 107 | 124 |
| InfA_TH5xR14 | gTggATTCTTTgTCTgCAgCg T A | -14 | -2 | 109 | 109 |
| InfA_TH5xR** | gTggATTCTTTgTCTgCAgCg XT A | -2.9 | -2 | 100 | 180 |

### Influenza H5N1 Amplification N1 Gene

| | | | | |
|---|---|---|---|---|
| InfAN1_F | AggAATgCTCCTgTTATCC XT gA | S | 312-333 | 55.7° C. |
| InfAN1_R | gCCgTATTTAAATgAAAACCC XT T | A | 544-522 | 56.6° C. |

### Amplification HIV-1 Gag Gene Region

| | | | | |
|---|---|---|---|---|
| SKCC1B+^{MB} | ggTACTAgTAgTTCCTgCTATgTCACTTCC | A | 1515-1486 | 59.7° C. |
| SKCC1B | TACTAgTAgTTCCTgCTATgTCACTTCC | A | 1513-1486 | 56.2° C. |

### Amplification HCV 5-UTR Region

| [xx] | HCV 5-UTR | | D10934 | Tm |
|---|---|---|---|---|
| KY80 | gCAgAAAgCgTCTAgCCATggCgT | S | 68-91 | 68.0° C. |
| KY80n_bi | XCAgAAAgCgTCTAgCCATggCgT | S | 69-91 | 65.4° C. |
| KY78 | CTCgCAAgCACCCTATCAggCAgT | A | 311-288 | 66.3° C. |
| KY78n | TCgCAAgCACCCTATCAggCAgT | A | 310-288 | 65.5° C. |
| Mblau | CTCgCAAgCACCCTATCAggCAg XT A | A | 311-289 | 66.3° C. |

1-15. (canceled)

16. A method for enzymatic amplification of nucleic acid sequences, wherein
in a reaction step preceding the amplification, an enzymatic reverse transcription activity is used in said reaction step for polymerization of deoxyribonucleic acid from a ribonucleic acid template, and where
at least three different primers are used, each of which comprises a sequence that can be described by the formula 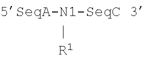
wherein
the sequence SeqA-N1-SeqC has a length of at least 8 nucleotides and is able to hybridize with one of the nucleic acid sequences to be amplified,
SeqA and SeqC are sequences of naturally occurring deoxyribonucleic elements or their synthetic analogs,
SeqA 0 comprises at least 40 nucleotides,
SeqC represents a sequence of 1 to 20 nucleotides in length,
N1 is a phosphoribonucleic moiety covalently bound to R1 or a nucleotide analog covalently bound to R1,
R1 is a molecular moiety comprising ten atoms of an atomic weight of 12 or more; and
R1 comprises two to five rings with four to seven atoms as ring members per ring.

17. The method according to claim 16, wherein at least four different primers are used for the amplification of at least two different nucleic acid sequences.

18. The method according to claim 16, wherein R1 comprises at least fifteen atoms of an atomic weight of twelve or more.

19. The method according to claim 16, wherein R1 is methylene blue or methyl orange.

20. A method for enzymatic amplification of nucleic acid sequences, wherein
in a reaction step preceding the amplification, an enzymatic reverse transcription activity is used in said reaction step for polymerization of deoxyribonucleic acid from a ribonucleic acid template, and
more than two synthetic oligodeoxynucleic acid compounds are used as primers, and in which at least one of said primers comprises at least one sequence that can be described by the formula 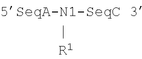
the sequence SeqA-N1-SeqC has a length of at least 8 nucleotides and is able to hybridize with one of the nucleic acid sequences to be amplified,
SeqA and SeqC are sequences of naturally occurring deoxyribonucleic elements or their synthetic analogs,
SeqA 0 comprises at least 40 nucleotides,
SeqC represents a sequence of 1 to 20 nucleotides in length,
N1 is a phosphoribonucleic moiety covalently bound to R1 or a nucleotide analog covalently bound to R1; and
R1 is methylene blue or methyl orange.

21. A reaction mix for enzymatic amplification of nucleic acid sequences, comprising an enzymatic reverse transcription activity for polymerization of deoxyribonucleic acid from a ribonucleic acid template, and more than two synthetic oligodeoxynucleic compounds as primers, where at least three of said primers comprises at least one sequence, which can be described by the formula 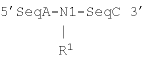
wherein
the sequence SeqA-N1-SeqC has a length of at least 8 nucleotides and is able to hybridize with one of the nucleic acid sequences to be amplified,
SeqA and SeqC are sequences of naturally occurring deoxyribonucleic elements or their synthetic analogs,
SeqA 0 comprises at least 40 nucleotides,
SeqC represents a sequence of 1 to 20 nucleotides in length,
N1 is a phosphoribonucleic moiety covalently bound to R1 or a nucleotide analog covalently bound to R1,
R1 is a molecular moiety comprising ten atoms of an atomic weight of 12 or more; and
R1 comprises two to five rings with four to seven atoms as ring members per ring.

22. The reaction mix according to claim 21, wherein the reaction mix comprises at least four different primers for amplification of at least two different nucleic acid sequences.

23. The reaction mix according to claim 21, wherein at least one of the following requirements is fulfilled:
R1 comprises at least fifteen atoms of a atomic weight of twelve or more;
R1 is methylene blue or methyl orange.

24. A reaction mix for enzymatic amplification of nucleic acid sequences, comprising an enzymatic reverse transcription activity for polymerization of deoxyribonucleic acid from a ribonucleic acid template, and more than two synthetic oligodeoxynucleic compounds as primers, where at least one of said primers comprises at least one sequence, which can be described by the formula 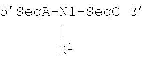
wherein
the sequence SeqA-N1-SeqC has a length of at least 8 nucleotides and is able to hybridize with one of the nucleic acid sequences to be amplified,
SeqA and SeqC are sequences of naturally occurring deoxyribonucleic elements or their synthetic analogs,
SeqA 0 comprises at least 40 nucleotides,
SeqC represents a sequence of 1 to 20 nucleotides in length,
N1 is a phosphoribonucleic moiety covalently bound to R1 or a nucleotide analog covalently bound to R1; and
R1 is methylene blue or methyl orange.

25. A primer mix for use in a method according to claim 16 comprising at least three primers comprising a sequence that can be described by the following formula 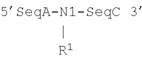
wherein
the sequence SeqA-N1-SeqC has a length of at least 8 nucleotides and is able to hybridize with one of the nucleic acid sequences to be amplified,
SeqA and SeqC are sequences of naturally occurring deoxyribonucleic elements or their synthetic analogs,
SeqA 0 comprises at least 40 nucleotides,
SeqC represents a sequence of 1 to 20 nucleotides in length,
N1 is a phosphoribonucleic moiety covalently bound to R1 or a nucleotide analog covalently bound to R1; and
R1 is methylene blue or methyl orange.
